(19) United States
(12) Patent Application Publication
(10) Pub. No.: US 2023/0071076 A1
(43) Pub. Date: Mar. 9, 2023

(54) PROSTHETIC TEAR DUCT DEVICES

(71) Applicant: Jonah Adler, Atlanta, GA (US)

(72) Inventor: Jonah Adler, Atlanta, GA (US)

(21) Appl. No.: 17/901,639

(22) Filed: Sep. 1, 2022

Related U.S. Application Data
(60) Provisional application No. 63/260,841, filed on Sep. 2, 2021.

(51) Int. Cl. A61F 9/007

(52) U.S. Cl. CPC A61F 9/00772; A61F 2220/0016; A61F 2240/001

(57) ABSTRACT

Prosthetic devices described herein include a body comprising a first end portion and a second end portion. The body defines a longitudinal lumen extending between the first and second end portions. In some embodiments, one or more anchor members are movably coupled to the body. Each anchor member can be selectively reconfigurable between a radially retracted position and a deployed position in which tip end portions of the one or more anchor members extend radially outward from an outer surface of the body. In some embodiments, a one-way valve is located within, and/or is otherwise in fluid communication with, the longitudinal lumen.

### CROSS-REFERENCE TO RELATED APPLICATION

The application claims the benefit of priority to U.S. Provisional Patent Application No. 63/260,841 filed on Sep. 2, 2021, the entire contents of which are hereby incorporated by reference.

### FIELD OF THE INVENTION

The devices and methods disclosed herein relate to implantable/insertable medical devices for creating/maintaining an artificial connection between bodily regions of one or more organs, including, but not limited to, the eye, nose, throat, mouth, ear, sinuses, and their surrounding anatomic structures. For example, this disclosure relates to prosthetic tear duct tubes.

### BACKGROUND

The lacrimal drainage system, commonly known as the “tear duct” removes tears from the surface of the eye. It consists of the puncta, canaliculi, lacrimal sac, and nasolacrimal duct. Patients who develop obstruction of the tear ducts experience severe, bothersome tearing and infection. Obstructions may develop due to normal aging changes, infection, trauma, tumor, or on a congenital basis. A significant subset of lacrimal drainage problems are due to obstruction of the canaliculi, which usually cannot be surgically reconstructed. Such patients require a conjunctivodacryocystorhinostomy (“CDCR”). This procedure creates an artificial tear duct by drilling through the bone of the lacrimal fossa and placing a PYREX® tube that directly connects the medial canthus to the nasal cavity, essentially bypassing the malfunctioning lacrimal drainage system and permitting tears to drain into the nose. This device can be removed and replaced in-office or in the operating room using the Seldinger or alternative surgical techniques. These devices, commonly referred to as a Jones tube, have undergone numerous modifications due to significant problems with the implantation of a relatively large foreign body in this sensitive area (e.g., Putterman-Gladstone, Callahan-Cox, Straiko modified Jones tube, LEITR frosted Jones tube, Medpor-coated Jones tube, Stop-Loss Jones tube, angled Jones tubes, incorporation of suture holes and/or double-flanged tubes). Each design has its own pros and cons, but all options currently have functional deficiencies.

For example, the tubes described above are plagued by problems of dislocation and extrusion. Dislocation and extrusion usually necessitate a repeat surgical procedure under anesthesia, incurring significant additional cost and potential surgical morbidity.

A problem that no design modification has been able to address is reflux of air and secretions from the nasal passage to the eye region. This is because, unlike the natural nasolacrimal system, which is complete with a series of valves to prevent reflux, the PYREX tubes are essentially a “two-way street.” This is especially problematic for patients who use continuous positive airway pressure (“CPAP”) for obstructive sleep apnea or supplemental oxygen for other pulmonary conditions. Such patients are poor candidates for Jones tubes due to the very bothersome air and mucus that tend to reflux onto the ocular surface from the nasal cavity.

There exists a need for new approaches to address the problems described above (e.g., to address reflux of air and secretions from the nasal passage to the eye).

### SUMMARY

In general, one aspect of this document features a prosthetic tear duct tube device having a hollow cylindrical body with openings at each end. The ends can include one or more flanges. The hollow cylindrical body can contain, and/or can be in fluid communication with, a unidirectional valve (e.g., a one-way valve). The prosthetic tear duct tube device can include anchor members. In some embodiments, the anchor members are radially extendable and/or retractable anchor members.

In one aspect, the devices, methods, and systems disclosed herein include a prosthetic tear duct system, having a body comprising first end portion and a second end portion, the body defining a longitudinal lumen extending between the first and second end portions; one or more anchor members movably coupled to the body, each anchor member being selectively reconfigurable from a retracted position to a deployed position, where tip end portions of the one or more anchor members extend radially outward from an outer surface of the body when the one or more anchor members are in the deployed positions, and where, when the one or more anchor members are in the retracted positions, the one or more anchor members are each radially retracted in comparison to the deployed positions; and a one-way valve within the longitudinal lumen.

In some embodiments, the one-way valve is one of a shut-off valve, a ball-and-spring-valve, a compression stop valve, a compression stop valve, swing check valve, tilting disc check valve, reed valve, diaphragm check valve, or a duckbill valve. In some embodiments, the one-way valve is configured to allow passage of fluid through the longitudinal lumen in a first direction from the first end portion to the second end portion and to prevent fluid from passing through the longitudinal lumen in a second direction from the second end portion to the first end portion. As used herein, fluid can include liquid and/or gas (e.g., air, CO₂, O₂, etc.).

In some embodiments, the one or more anchor members having a plurality of anchor barbs that are individually selectively reconfigurable from the retracted position to the deployed position. In some embodiments, the plurality of anchor barbs are individually selectively reconfigurable from the deployed position to the retracted position.

In some embodiments, the system can include a tool configured for manual reconfiguration of the plurality of anchor barbs between the deployed positions and the retracted positions. In some embodiments, the system can include a valve-housing unit having the one-way valve within the longitudinal lumen. In some embodiments, the body is a cylindrical tube, an oval tube, a conical tube, a curved tube, or a polygon prism. In some embodiments, the longitudinal lumen has an hourglass shape. In some embodiments, the first end portion of the body of the prosthetic tear duct apparatus includes a flange.

Another aspect of devices, systems, and methods disclosed herein include a prosthetic tear duct apparatus having a body having a first end portion and a second end portion, the first end portion having a first flange, the body defining a longitudinal lumen extending between the first and second end portions; and a plurality of rings extending from an outer surface of the body and configured to contact nasolacrimal tissue, where the longitudinal lumen has an hourglass shape.

In some embodiments, the apparatus can further include a one-way valve within the longitudinal lumen. In some embodiments, the one-way valve is one of one of a shut-off valve, a ball-and-spring-valve, a compression stop valve, a compression stop valve, swing check valve, tilting disc check valve, reed valve, diaphragm check valve, or a duckbill valve. In some embodiments, the apparatus can further include a valve-housing unit proximal to the second end portion of the body configured to contain the one-way valve, where the one-way valve is configured to allow passage of fluid through the longitudinal lumen in a first direction from the first end portion to the second end portion and prevent fluid from passing through the longitudinal lumen in a second direction from the second end portion to the first end portion.

In some embodiments, the second end portion includes a second flange. In some embodiments, the second flange has a smaller circumference than the first flange. In some embodiments, the apparatus can further include a selectively removable plug that is configured to couple to the first end portion, the second end portion, or both.

Another aspect of devices, systems, and methods described herein include a method of manufacturing a tear duct apparatus, including forming a tube body having a first end portion and a second end portion, the first end portion having a first flange, the tube body defining a longitudinal lumen extending between the first and second end portions; and a plurality of rings extending from an outer surface of the tube body and configured to contact nasolacrimal tissue, where the longitudinal lumen has an hourglass shape.

In some embodiments, the tube body is made of glass, metal, plastic, rubber, latex, or combinations thereof. In some embodiments, the tube body includes a frosted coating, a microporous high-density polyethylene implant coating, or both.

Some embodiments of the devices, systems, and methods described herein may provide one or more of the following advantages. First, in some embodiments anchor members described herein can be selectively reconfigurable between radially retracted positions and radially extended positions (also referred to as deployed positions). The selectively reconfigurable anchor members can be deployed and retracted ad hoc. This can allow the insertion and removal of the prosthetic tear duct tube device as needed for repositioning, cleaning, treatment, and mitigation of ailments that are corrected by the device. In some examples, the anchor members are selectively deployable to secure the tube body in place in a region, preventing migration and/or extrusion.

Second, in some embodiments a one-way valve positioned within, and/or is otherwise in fluid communication with, the longitudinal lumen of the prosthetic tear duct tube device can be included to prevent the reflux of fluids from a first region to a second region. For example, a one-way valve positioned within the longitudinal lumen can prevent reflux of fluids from the nasal passage to the eye region. This can be particularly helpful to patients that utilize devices such as CPAP machines, utilize other positive air pressure treatments, or who smoke.

Third, in some embodiments flanges can be positioned proximate to or at one or both end portions of the tube body to help secure the prosthetic tear duct tube device in place at the implant site. In some examples, the flanges can be selectively deployable to secure the tube body in place, preventing migration and/or extrusion.

As used herein, the term “substantially” infers that the characteristic needs not be absolute, but is close enough so as to achieve the advantages of the characteristic. For example, “substantially perpendicular” is not limited to absolute perpendicularity, and can include orientations that are intended to be parallel but due to manufacturing limitations may not be precisely perpendicular. For example, “substantially perpendicular” features are at least closer to a perpendicular orientation than a parallel orientation, e.g., within a few degrees of perpendicular such as +/−2°, +/−5°, or +/−10°. Similarly, “substantially parallel” is not limited to absolute parallelism, and can include orientations that are intended to be parallel but due to manufacturing limitations may not be precisely parallel. For example, “substantially parallel” features are at least closer to a parallel orientation than a perpendicular orientation, and generally are formed within a few degrees of parallel such as +/−2°, +/−5°, or +/−10°.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIG. 1 presents a view of the location of an example prosthetic tear duct device that is implanted within a patient to act as a tear duct.

FIG. 2 presents an oblique view of the outside of the device of FIG. 1.

FIG. 3 presents a side view of the outside of the device of FIG. 1.

FIG. 4 presents a longitudinal cross-section of the device of FIG. 1.

FIG. 5 presents a longitudinal cross-section of the device of FIG. 1 equipped with a flange on a second end portion.

FIG. 6 presents a lengthwise cross-section of the device of FIG. 1 equipped with a flange proximate to the second end portion.

FIG. 7 presents a top view of the device of FIG. 1, with anchor members in deployed position.

FIG. 8 presents a top view of the device of FIG. 1, with anchor members in a retracted position.

FIG. 9 presents a perspective view of an example anchor member deployment tool.

FIG. 10A presents a perspective view of an example anchor member retraction tool.

FIG. 10B presents an enlarged view of a tip portion of the anchor member retraction tool of FIG. 10A.

FIG. 11 presents an oblique view of the outside of another example prosthetic tear duct device.

FIG. 12 presents a longitudinal cross-section of the device of FIG. 11 defining an irregular shaped longitudinal lumen.

FIG. 13 presents a longitudinal cross-section of the device of FIG. 11 defining a uniformly shaped longitudinal lumen.

FIG. 14 presents an exemplary method of manufacturing a prosthetic tear duct device.

In the figures, like reference numbers indicate like elements throughout.

### DETAILED DESCRIPTION

The devices, such as the prosthetic tear duct devices, described herein can be used in, or to connect, one or more organs or anatomic regions to each other or to surrounding regions, organs, or anatomic structures, including the eye, nose, throat, mouth, ear, sinuses, and their surrounding anatomic structures. The devices described herein can include all or any combination of features described below.

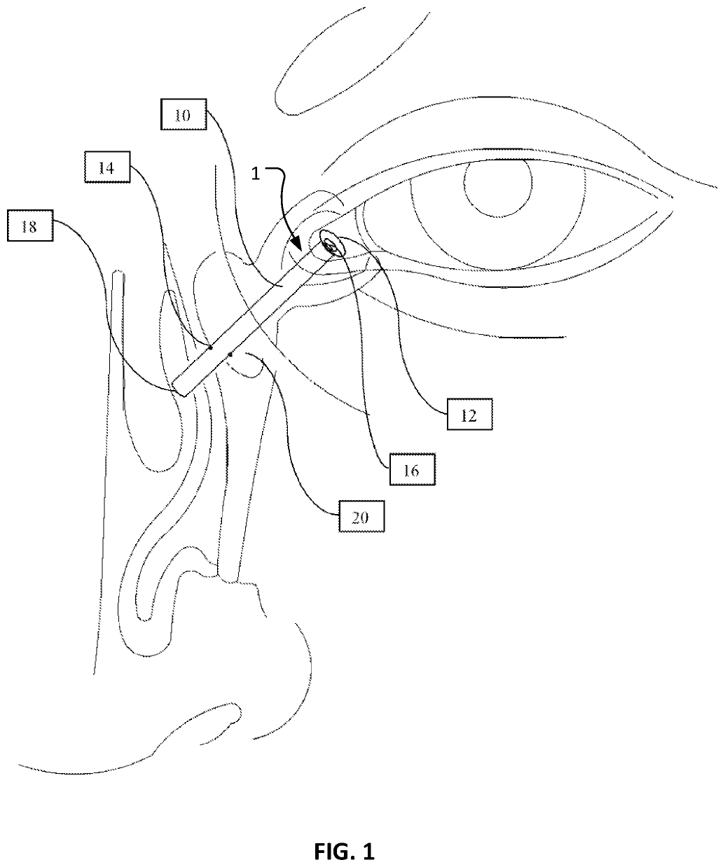

FIG. 1 presents a view of an example prosthetic tear duct device 1 when implanted within nasolacrimal structures 20 (e.g., to act as a tear duct). In this orientation, the prosthetic tear duct device 1 directly connects the medial canthus to the nasal cavity so that excess tear fluid drains to the nasal cavity. The prosthetic tear duct device 1, when implanted as shown, can alleviate a blocked tear duct condition.

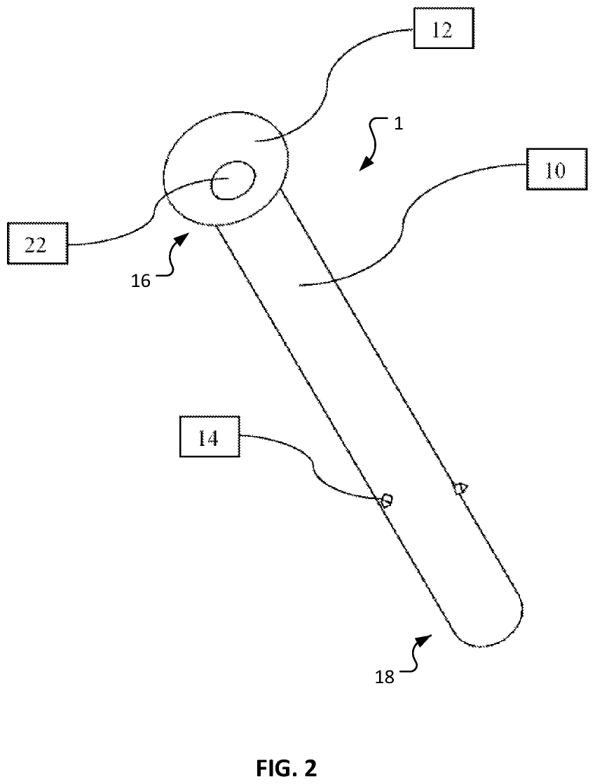

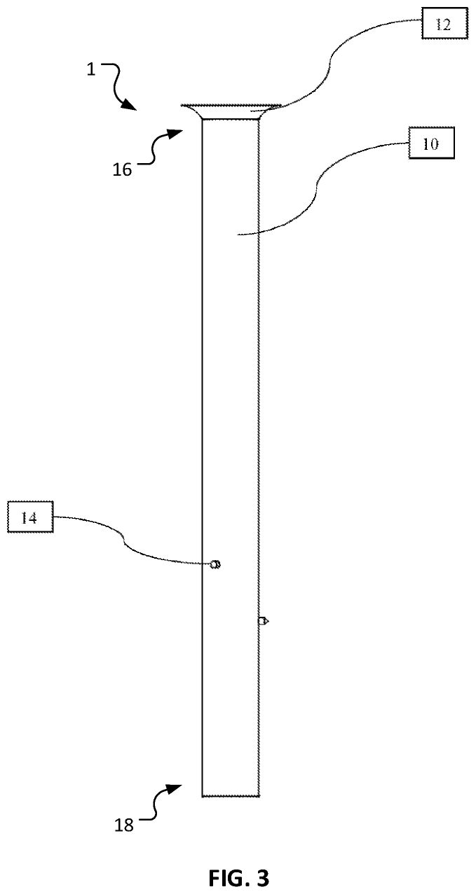

Referring also to FIGS. 2-4, the prosthetic tear duct device 1 includes a tube body 10 that defines a lumen 22. The tube body 10 has a first end portion 16 and a second end portion 18. In the depicted embodiment, the first end portion 16 includes an external flange 12. The prosthetic tear duct device 1 also includes one or more selectively reconfigurable anchor members 14 and a one-way valve 25, as described further below.

According to an embodiment, the tube body 10 comprises a hollow body forming a longitudinal lumen 22 therein with openings at each end (e.g., the first end portion 16 and second end portion 18).

The example prosthetic tear duct device 1 includes a tube body 10 having a cylindrical shape. The tube body 10 can be a rigid structure or a flexible structure. For example, the tube body can be made of glass, metal, plastic, rubber, latex, silicone, or any combination thereof. In some embodiments, the tube body 10 can include a coating. For example, the tube body 10 can include a frosted coating, a microporous high-density polyethylene implant coating, or both.

The length of the tube body 10 can be in a range of between about 5 mm to about 25 mm. For example, the length of the tube body 10 can be about 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, or 25 mm. The length of the tube body 10 can be based on the length of the anatomy in which the prosthetic tear duct device 1 will be placed. For example, a patient can be sized for a particular appropriate length. The length of the hole/tunnel drilled from the medial canthus of the eye into the nasopharynx helps the surgeon determine what length of tube body 10 will be needed.

The outer diameter of the tube body 10 can be in a range of between about 1 mm to about 3 mm. For example, the diameter of the tube body 10 can be about 1 mm, 1.25 mm, 1.5 mm, 1.75 mm, 2 mm, 2.25 mm, 2.5 mm, 2.75 mm, or 3 mm. The selected outer diameter of the tube body 10 can depend on the diameter of drill-bit used to create the hole/tunnel.

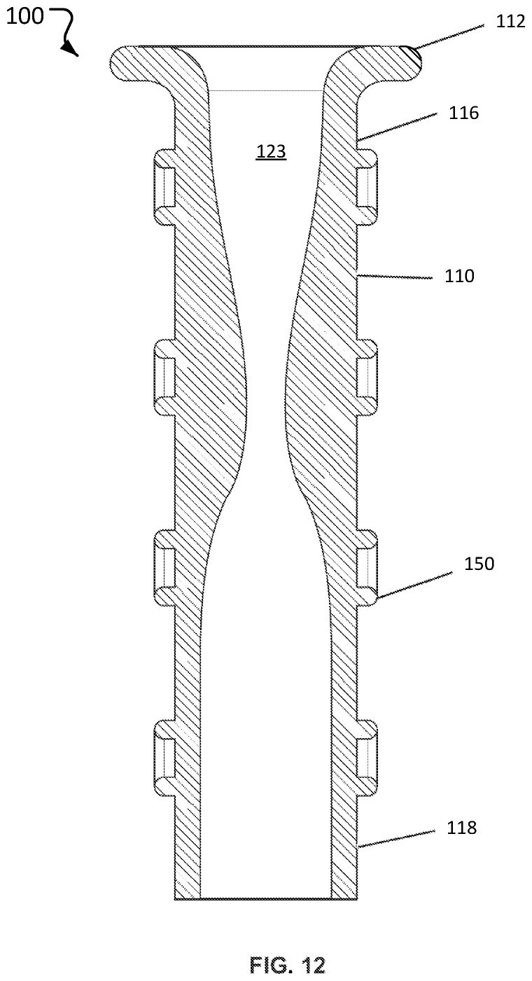

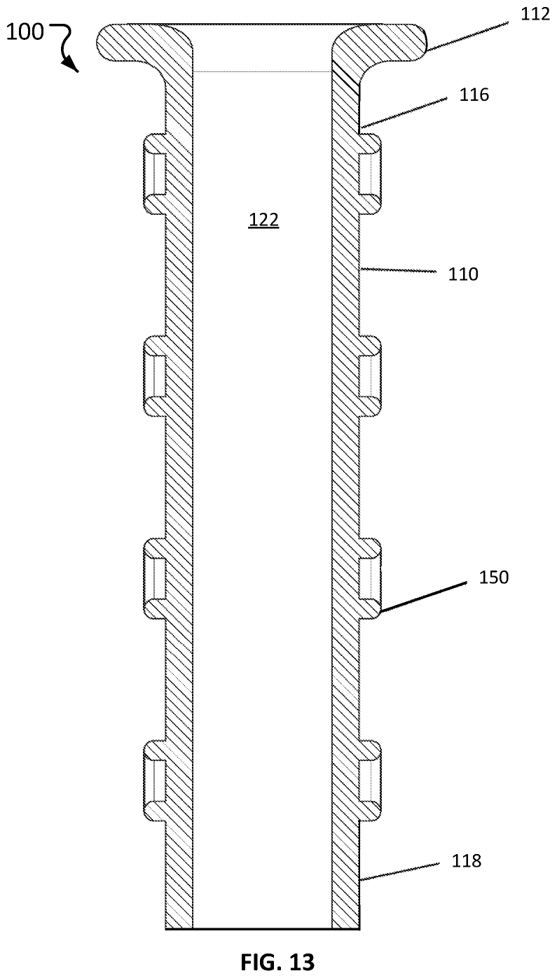

According to an embodiment, the tube body 10 comprises a hollow body forming a longitudinal lumen 22 therein with openings at the first end portion 16 and second end portion 18. The longitudinal lumen 22 can extend between the first end portion 16 and the second end portion 18 of the tube body 10. In the embodiment depicted, the longitudinal lumen 22 can have a cylindrical shape with walls that are substantially straight and/or on planes that are substantially parallel. The inner diameter of the tube body 10 defining the lumen 22 can be in a range of between about 1.0 mm to about 2.0 mm. For example, the inner diameter of the tube body 10 can be about 1.0 mm, 1.1 mm, 1.2 mm, 1.3 mm, 1.4 mm, 1.5 mm, 1.6 mm, 1.7 mm, 1.8 mm, 1.9 mm, or 2 mm. The interior diameter of the tube body 10 can vary based on the material of the tube body 10. In some embodiments, the interior diameter of the tube body 10 can vary along the length of the tube body 10, depending on a shape of the longitudinal lumen 22 (e.g., as shown in FIG. 12).

In the depicted embodiment, the first end portion 16 (e.g., the end near the medial canthus of the eye when the device 1 is implanted to act as a prosthetic tear duct) includes the external flange 12 surrounding and radially extending substantially perpendicular to the tube body 10. The external flange 12 at the first end portion 16 of the tube body 10 can fluidly communicate (e.g., contact) with the opening of the canal in the region of the lower punctum and medial canthus of the eye.

The external flange 12 can be positioned on either the first end portion 16 and/or the second end portion 18 of the tube body 10, and can extend beyond the outside diameter of the tube body 10 (e.g., by up to 0.5 mm radially). In some examples, the external flange 12 can be composed of an inflatable balloon to assist with keeping the prosthetic tear duct device 1 stationary after implantation.

Any suitable manner of anchoring the tube body 10 to an anatomical region (e.g., the wall of an anatomical region) shall be included in the scope of this disclosure. For example, the depicted anchor members 14 can be configured to secure the tube body 10 to the tissue of the anatomical region (e.g., tunnel or cavity) in which it is placed. One or more of the anchor members 14 can be included as part of the prosthetic tear duct device 1. For example, some embodiments of the prosthetic tear duct device 1 include one, two, three, four, five, six, seven, eight, nine, ten, or more than ten of the anchor members 14. Non-limiting examples of anchor members 14, in addition to the depicted anchor members 14, include a projection (e.g., a barb), a helical screw, a curved barb, a hooked barb, a shaped protrusion (e.g., including ridges, circles, and/or textured radial lines), a balloon (when inflated can take on any of the anchor member shapes/contours listed), or splitting barbs (one barb base splits to create multiple branches).

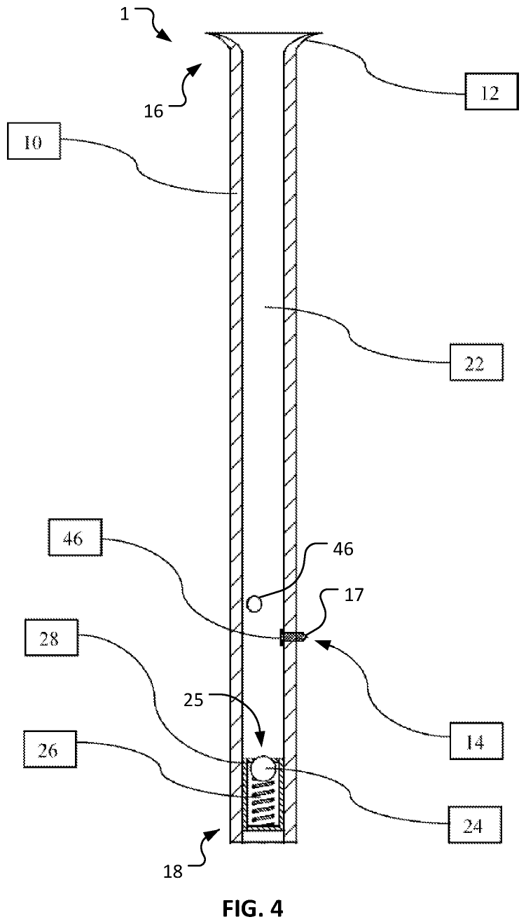

As best seen in FIG. 4, the depicted example prosthetic tear duct device 1 includes the anchor member 14 that is a projection with a base 46 that extends from the longitudinal lumen 22 to the external portion of the tube body 10. The anchor member comprises a tip portion 17. As shown, the anchor member 14 can be deployed to extend radially from the external surface of the tube body 10. In some examples, an anchor member 14 can be deployed to traverse the tube body 10 such that the base 46 of the anchor member 14 is accessible in the longitudinal lumen 22 while at least the tip portion 17 extends radially outward from the exterior of the tube body 10.

The anchor member 14 can be of any shape and/or positioned at any angle relative to the external portion of the tube body 10. For example, the anchor member 14 can be linear and deployed purely radially such that it is substantially perpendicular to a longitudinal axis of the tube body 10. In another example, an anchor member 14 can extend outward from the tube body 10 at an angle that is anywhere between 0° and 90° relative to the longitudinal axis of the tube body 10. In some embodiments that include multiple anchor members 14, at least one anchor member 14 can extend in a direction that is at least partially opposite to another anchor member 14. For example, one anchor member 14 can be angled towards the first end portion 16 and another anchor member 14 can be angled towards the second end portion 16.

In some embodiments, the one or more anchor members 14 can be selectively reconfigurable. For example, an anchor member 14 can be moveably coupled to the tube body 10 and selectively reconfigurable from a retracted position to a deployed position. In one example embodiment, two or more anchor members 14 can be deployed simultaneously. In another example, each or the one or more anchor members 14 can be deployed independently from the other one or more anchor members 14. For example, a first anchor member 14 can be radially deployed (outward from the tube body 10) while a second anchor member 14 is remains radially retracted. In another example, a first anchor member 14 and a second anchor member 14 can be deployed simultaneously.

In another aspect, in some embodiments the one or more anchor members 14 can be retracted independently from one or more other anchor members 14. That is, a first anchor member 14 can be retracted while a second anchor member 14 remains radially deployed. In another example, a first anchor member 14 and a second anchor member 14 can be retracted simultaneously.

The prosthetic tear duct device 1 described herein can include a varying quantity of anchor members 14, anchor member 14 can vary in orientation (direction pointing when deployed), anchor member 14 can vary in locations along the tube body 10 (e.g., on the first end portion 16, on the second end portion 18, and/or anywhere there between).

The anchor member 14 can also vary in shape (e.g., shape-straight, hooked, curved, splitting, etc.). An anchor member 14 can be made from a variety of materials such as a single metal or a combination of metals, resins, plastics, glass, or other composites. The anchor member 14 can be deployed through individual holes in the tube body 10. In some embodiments, multiple anchor members 14 can be deployed through an individual hole in the tube body 10.

In the depicted embodiment of the prosthetic tear duct device 1, the one-way valve 25 is located in the longitudinal lumen 22 to prevent reflux (retrograde fluid flow) as described above. That is, the one-way valve 25 is configured to allow passage of fluid through the longitudinal lumen 22 in a first direction (from the first end portion 16 to the second end portion 18) and to prevent fluid from passing through the longitudinal lumen 22 in a second, opposite direction (from the second end portion 18 to the first end portion 16).

The depicted one-way valve 25 includes a housing 28, a spring 26, and a valve member 24. The spring 26 and the valve member are disposed within an internal space defined by the housing 28. The spring 26 biases the valve member 24 to its closed position as shown, in which fluid cannot flow through the one-way valve 25. The spring force of the spring 26 can be overcome, however, by a fluid pressure acting on the valve member 24. In such a case, the valve member 24 can compress the spring 26 and move from its closed position to an open position that allows fluid to flow through the one-way valve 25 toward the second end portion 18. The one-way valve 25 can be positioned within the lumen 22 proximate to the first end portion 16 of the tube body 10, proximate to the second end portion 18 of the tube body 10, or anywhere there between. In some embodiments, the one-way valve 25 can be located at either end of the tube body 10 (e.g., external to either of the openings at the first end portion 16 or the second end portion 18).

While the one-way valve 25 is depicted as a ball-and-spring valve, other non-limiting examples of one-way valves that can be used include a shut-off valve, a compression stop valve, swing check valve, tilting disc check valve, reed valve, a duckbill valve, or, a diaphragm check valve.

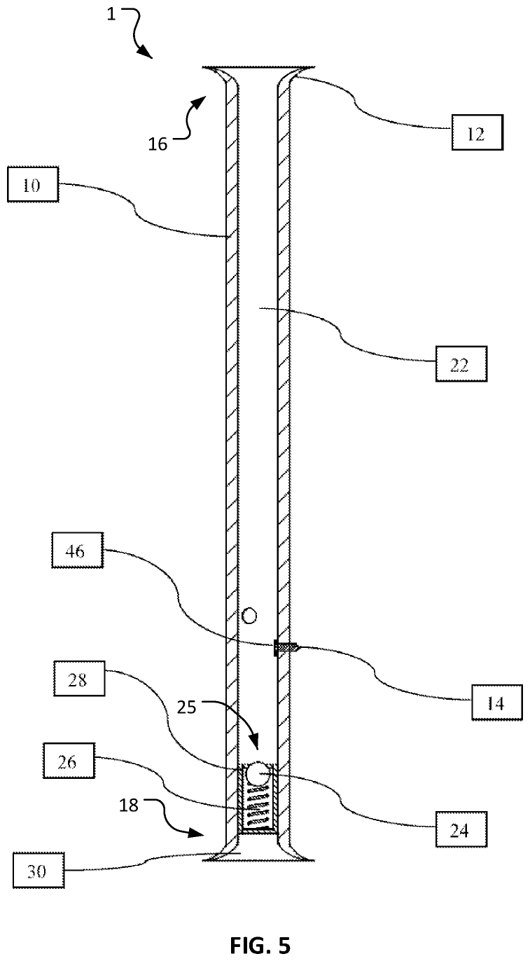

FIG. 5 presents a longitudinal cross-section of the prosthetic tear duct device 1 being additionally equipped with a flange 30 at the second end portion 18. That is, the prosthetic tear duct device 1 includes a first external flange 12 at the first end portion 16, and a second external flange 30 at the second end portion 18.

In the depicted embodiment, the second end portion 18 of the tube body 10 includes the second flange 30 which is oriented substantially perpendicular to the longitudinal axis of the tube body 10. The flange 30 can be near or at the second end portion 18. In some embodiments, the flange 30 can be a flexible structure, capable of bending to allow for insertion and removal of the prosthetic tear duct device 1 with subsequent resumption of an erect shape following an insertion or removal action. The flange 30 on the second end portion 18 can fluidly communicate (e.g., contact) with the opening of the anatomical region (e.g., on the nasal/oropharynx tissue). In some embodiments, the circumference of the flange 30 can have a larger circumference than the external flange 12. In some embodiments, the flange 30 can have a circumference that is smaller than the external flange 12. In some embodiments, the flange 30 can have a circumference that is substantially the same as the external flange 12.

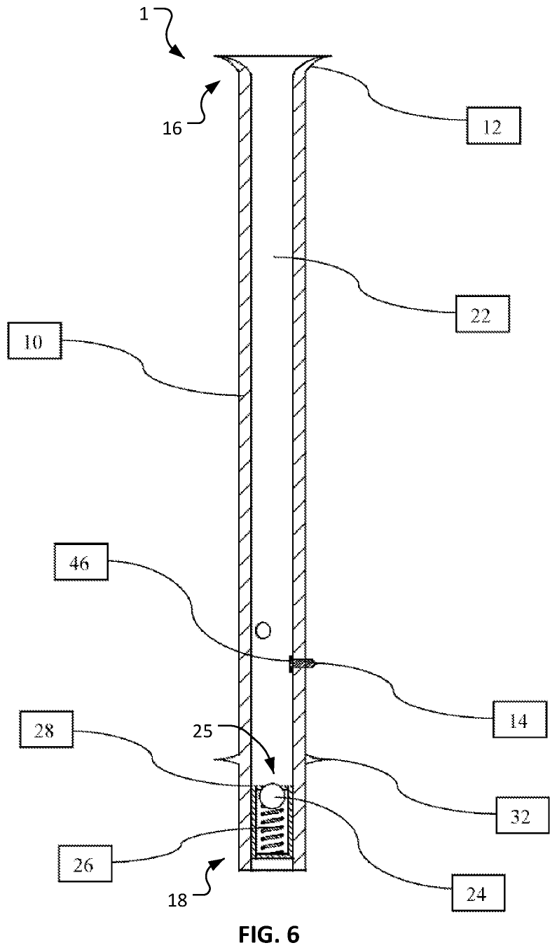

FIG. 6 presents a lengthwise cross-section of the prosthetic tear duct device 1 equipped with a second flange 32 located proximate to the second end portion 18. The depicted prosthetic tear duct device 1 includes the first external flange 12 at the first end portion 16, and the second external flange 32 proximate to the second end portion 18.

As shown in FIGS. 5 and 6, in some embodiments the prosthetic tear duct device 1 can include multiple flanges. For example, the prosthetic tear duct device 1 can include the external flange 12, the external flange 30, and/or the external flange 32. In some such embodiments, a circumference of the flange 32 can be larger than that of the external flange 12 and/or the flange 30. In some embodiments, the flange 32 can have a circumference that is smaller than the external flange 12 and/or the flange 30. In some embodiments, the flange 32 can have a circumference that is substantially the same as the external flange 12 and/or the flange 30.

In some embodiments, a flange (e.g., an external flange 12, flange 30, and/or flange 32) can be a balloon that can be inflated and deflated as needed for insertion or removal of the prosthetic tear duct device 1. For example, the flange 30 can be comprised of a balloon that can be in a deflated configuration when the prosthetic tear device 1 is inserted into an anatomical region and filled with fluid (e.g., a liquid or a gas) when in an inflated configuration to hold the prosthetic tear device 1 in place. In another example, the flange 32 can be comprised of a balloon that can be in a deflated configuration when the prosthetic tear device 1 is inserted into an anatomical region and filled with fluid when in an inflated configuration to hold the prosthetic tear device 1 in place. In another example, the flange 30 and the flange 32 can each be comprised of a balloon that can be in a deflated configuration when the prosthetic tear device 1 is inserted into an anatomical region and filled with fluid when in an inflated configuration to hold the prosthetic tear device 1 in place. In another example, the external flange 12, the flange 30, and the flange 32 can each be comprised of a balloon that can be in a deflated configuration when the prosthetic tear device 1 is inserted into an anatomical region and filled with fluid when in an inflated configuration to hold the prosthetic tear device 1 in place.

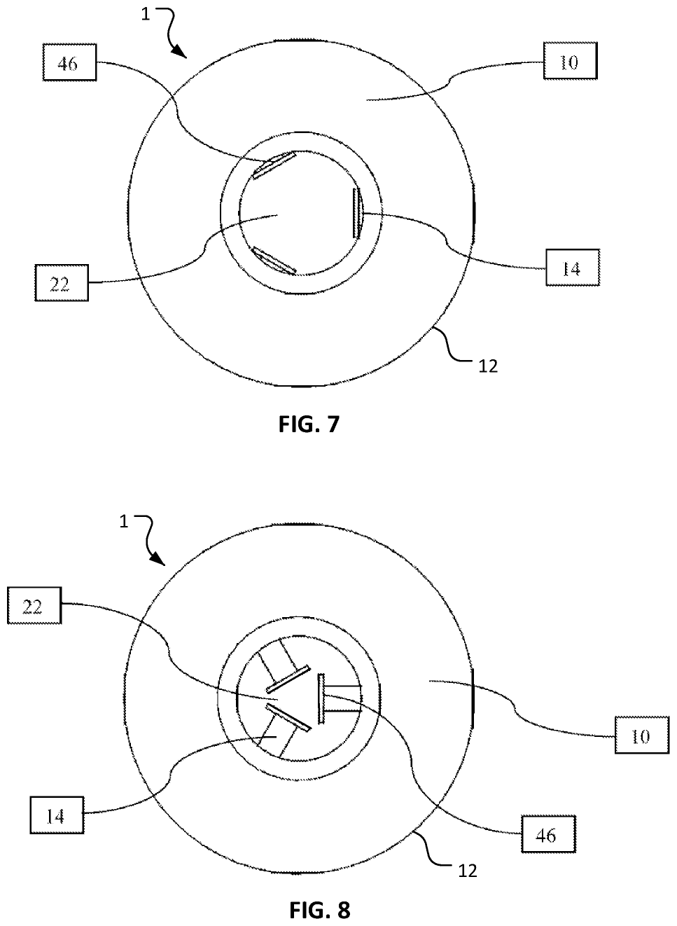

Referring also to FIGS. 7-8, these top views of the prosthetic device 1 show the anchor members 14 in the deployed positions (FIG. 7) and in the radially retracted positions (FIG. 8).

In some embodiments, the anchor members 14 can be individually selectively reconfigurable. For example, the one or more anchor members 14 can be movably coupled to the tube body 10, and each anchor member 14 can be selectively reconfigurable between the radially retracted position and the deployed position. In one example, the one or more anchor members 14 can be individually selectively reconfigurable from the retracted position to the deployed position (but not reconfigurable back to the retracted position). In another example, the one or more anchor members 14 can be individually selectively reconfigurable from the deployed position back to the retracted position.

The anchor members 14 in the deployed positions can secure the prosthetic tear duct device 1 such that the tube body 10 is fixed in the anatomical region (e.g., contacting the nasolacrimal tissue). For example, in the deployed position, the anchor member 14 can extend radially from the tube body 10 and contact nasolacrimal tissue to secure the prosthetic tear duct device 1 in place. Fixing the prosthetic tear duct device 1 in place can prevent dislocation and extrusion, while the selective reconfiguration from a deployed position to a retracted position can allow for removal when needed.

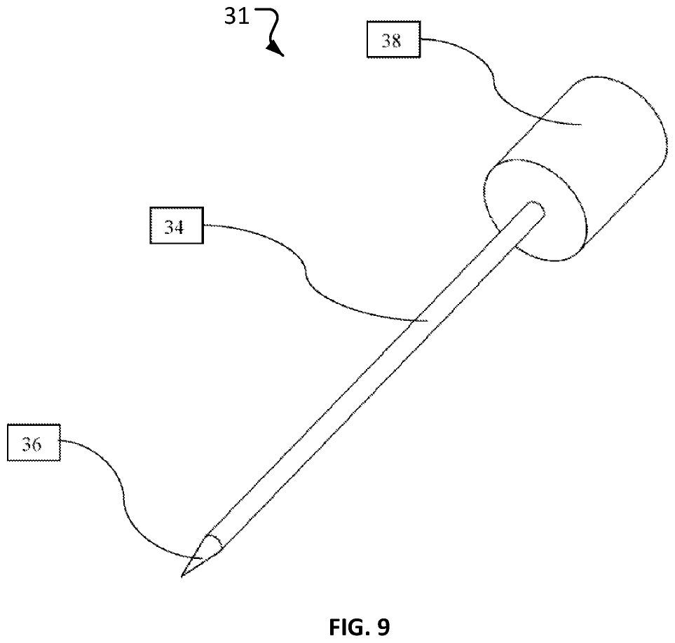

In one aspect, when the prosthetic tear duct device 1 includes an anchor member 14, a tip portion (e.g., tip portion 17; see FIG. 4) of the one or more anchor members 14 can extend radially outward from an outer surface of the tube body 10 when the one or more anchor members 14 are in deployed positions. The tip portion 17 of the anchor member 14 contacts (and/or pierces) the tissue of the anatomical region when in the deployed position. In some embodiments, the base 46 of the anchor member 14 is configured to be manually and selectively deployable when actuated by a tool (described in further detail in connection with FIG. 9).

The retracted position can release the prosthetic tear duct device 1 such that the tube body 10 is free to be removed and/or repositioned in anatomical region of which it is positioned (e.g., released from the nasolacrimal tissue). For example, in a retracted position, one or more of the anchor members 14 can be retracted radially from the tissue and into the tube body 10 such that the tip portion of the anchor member 14 prosthetic tear duct device 1 is no longer in contact with the anatomical region (e.g., the nasolacrimal tissue).

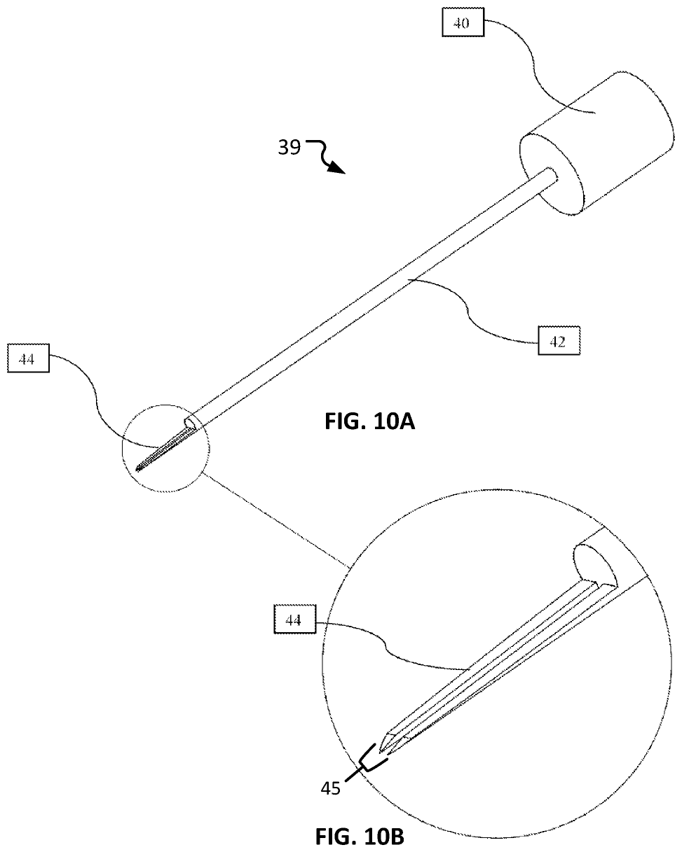

In some embodiments, the bases 46 of the anchor members 14 are configured to be manually and selectively retracted when actuated by a tool (described in further detail in connection with FIG. 10).

FIG. 9 presents a perspective view of an example anchor member deployment tool 31. The anchor member deployment tool 31 includes a handle 38, a shaft 34, and a tip 36. In use, the deployment of an anchor member (e.g., an anchor member 14) is performed by manually inserting the tip 36 and the shaft 34 into the longitudinal lumen (e.g., the longitudinal lumen 22) and pressing on a base (e.g., a base 46 of FIGS. 7 and 8) of one or more of the anchor members to deploy the anchor member(s) 14 to secure the prosthetic tear duct device 1 in an anatomical region (e.g., nasolacrimal tissue). In this example, the tip 17 of the one or more anchor members 14 is deployed by extending radially from the outer portion of the tube body 10 and contacting (e.g., piercing) the tissue of the anatomical region and securing the prosthetic tear duct device 1 in the anatomical region (e.g., nasolacrimal tissue).

FIG. 10A presents a perspective view of an example anchor member retraction tool 39. FIG. 10B presents an enlarged view of a tip 44 of the anchor member retraction tool 39. The anchor member retraction tool 39 includes a handle 40, a shaft 42, and the tip 44. As shown in FIG. 10B, the tip 44 includes fork mechanism 45. Selectively retracting one or more of the anchor members (e.g., an anchor member 14 of FIGS. 7 and 8) can be performed by manually using the anchor member retraction tool 39. In some embodiments, the shaft 42 of the anchor member retraction tool 39 is inserted in to the longitudinal lumen (e.g., longitudinal lumen 22) and the fork mechanism 45 of the tip 44 contacts one or more bases (e.g., base 46 of FIGS. 7 and 8) of the one or more anchor members and a force is applied toward the longitudinal lumen 22 to force the anchor member 14 radially inward to release the anchor member 14 from the anatomical region (e.g., nasolacrimal tissue).

For example, the retraction of one or more anchor members 14 can include sliding the fork mechanism 45 of the tip 36 around a base 46 of an anchor member 14 within the longitudinal lumen 22 and applying pressure to one or more of the anchor members 14 toward the internal portion of the longitudinal lumen 22. In this example, the tip 17 of the one or more anchor members 14 is retracted into the outer portion of the tube body 10 releasing the prosthetic tear duct device 1 from engagement with the anatomical region (e.g., nasolacrimal tissue).

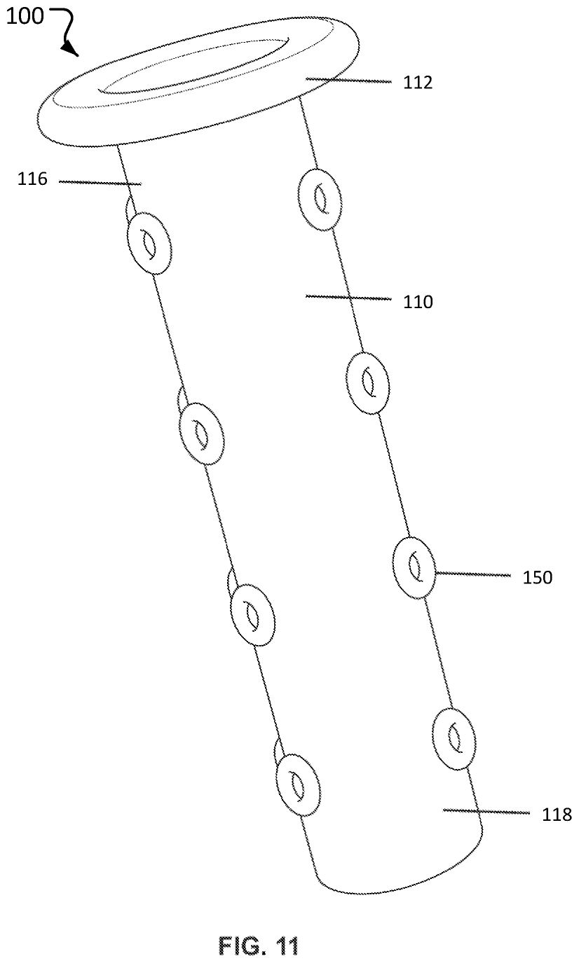

FIG. 11 is an oblique view of the outside of another prosthetic tear duct device 100. The prosthetic tear duct device 100 includes an external flange 112, a tube body 110, a first end portion 116, a second end portion 118, and a plurality of shaped protrusions 150. Though not shown in this example embodiment, the prosthetic tear duct device 100 of FIG. 11 can include a second flange located at the second end portion 118 and/or proximate to the second end portion 118 (e.g., in the positions of flange 30 of FIG. 5 or flange 32 of FIG. 6).

The shaped protrusions 150 of the depicted embodiment are toroidal (e.g., donut-shaped). However, the shaped protrusions 150 can be of any shape (e.g., rectangle, oval, eclipse, or any polygon). The shaped protrusion 150 protrudes from the outer surface of the tube body 110 and provides surface area configured to contact the tissue and provide a tight fit for the prosthetic tear duct device 100 within the anatomy. For example, a shaped protrusion 150 can be part of the external portion of the tube body 110 and configured to provide friction between the tube body 110 and the anatomical region (e.g., nasolacrimal tissue) of which the prosthetic tear duct device 100 is placed. The shaped protrusions 150 can be considered as raised portions of the external surface of the tube body 110. In one example, the shaped protrusion 150 can include raised circles that extend radially from the tube body 110.

FIG. 12 presents a longitudinal cross-section of the prosthetic tear duct device 100. Here it can be seen that the prosthetic tear duct device 100 defines an irregular shaped longitudinal lumen 123. That is, the diameter of the longitudinal lumen 123 differs at different locations along the tube body 110 between the first and portion 116 and the second end portion 118. The longitudinal lumen 123 can be of any shape. In the depicted embodiment, the longitudinal lumen 123 is hourglass-shaped, with the smallest diameter being at a location between the first end portion 116 and the second end portion 118. In some embodiments, the longitudinal lumen 123 can have another type of irregular shape, a cylindrical shape, or a polygon prism shape. Non-limiting examples of an irregular shape include a conical shape, an hourglass shape, or wave shape (e.g., a sine wave).

The longitudinal lumen 123 having an irregular shape can tend to help prevent fluid from flowing from the second end portion 118 toward the first end portion 116 and permit fluid to flow from the first end portion 116 toward the second end portion 118.

FIG. 13 presents a longitudinal cross-section of the prosthetic tear duct device 100 defining a uniformly shaped cylindrical longitudinal lumen 122. In some embodiments, longitudinal lumen 122 with the uniform shape can contain a one-way valve. For example, the longitudinal lumen 122 can include the one-way valve 25 (see FIG. 4).

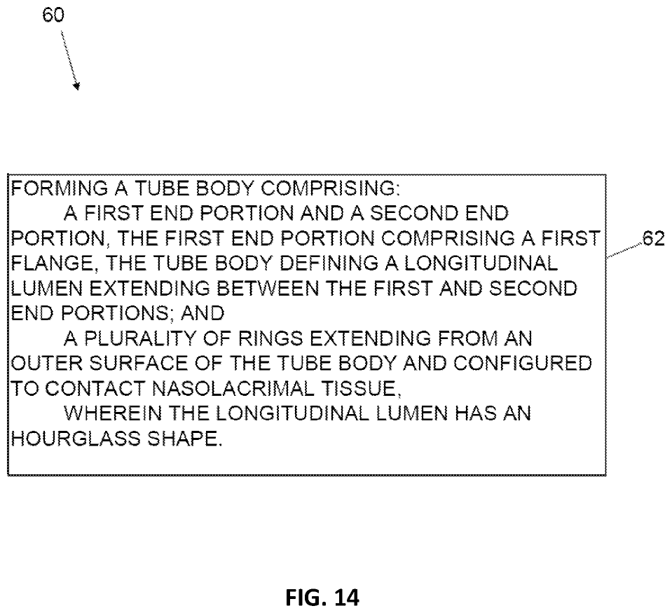

FIG. 14 describes an exemplary method 60 of manufacturing a prosthetic tear duct device (such as those described herein). The method 60 includes step 62 for forming a tube body. The forming method can include additive manufacturing, casting, silicone molding, pressure molding, articulated molding, blow molding, or piece-molding, handmade molding.

In some embodiments, a method of manufacturing a prosthetic tear duct device (e.g., prosthetic tear duct devices 1 or 100), can include forming a tube body (e.g., tube body 10 or 110) comprising a first end portion (e.g., first end portion 16 or 116) and a second end portion (e.g., second end portion 18 or 118), the first end portion 16 or 116 comprising a first flange (e.g., external flange 12 or 112), the tube body 10 or 110 defining a longitudinal lumen (e.g., longitudinal lumen 22, 122, or 123) extending between the first end portion 16 or 116 and second end portion 18 and 118 and one or more anchor members 14. In some embodiments, the anchor members 14 can be selectively reconfigurable. For example, the exemplary method of manufacturing a prosthetic tear duct device 1 or 100 can include forming one or more anchor members 14. The anchor members 14 can be formed to extend radially from the tube body 10 or 100 and contact the anatomical regions (e.g., nasolacrimal structures 20). In other embodiments, the method of manufacturing is an exemplary method of manufacturing a prosthetic tear duct device 1 or 100 that can include forming a shaped protrusion (e.g., shaped protrusion 150).

For example, in some embodiments the method 60 of manufacturing is an exemplary method of manufacturing a prosthetic tear duct device 100, and can include forming a plurality of rings (e.g., shaped protrusions 150) extending from an outer surface of the tube body 110 and configured to contact anatomical regions. In some embodiments, the method of manufacturing 60 a prosthetic tear duct device can include forming a longitudinal lumen 122 or 123. The longitudinal lumen can be formed in any shape. In some embodiments, the longitudinal lumen 123 can be of an irregular shape. For example, the longitudinal lumen 123 can be formed with an hourglass shape. In some embodiments, the method 60 of manufacturer can include forming or installing a valve 25. In some examples, the valve 25 is a one-way valve and can be formed within the longitudinal lumen 22, 122, or 123. In other examples, the one-way valve 25 can be formed proximate to the second end portion 18 or 118 of the tube body 10 or 110. The method of manufacturer of a tear duct apparatus 1 or 100 can include one or more of these features in any combination.

In one aspect, a method of manufacturing a tear duct apparatus 100, can include forming a tube body 110 comprising a first end portion the first end portion 116 and a second end portion the second end portion 118, the first end portion 116 comprising an external flange 112, the tube body 110 defining a longitudinal lumen 122 or 123 extending between the first end portion 116 and second end portion 118, and a plurality of shaped protrusions 150 extending from an outer surface of the tube body 110 and configured to contact an anatomical region. In some examples, the longitudinal lumen 123 has an hourglass shape. In other embodiments, the longitudinal lumen 122 has a uniform shape.

### OTHER FEATURES AND EMBODIMENTS

In some embodiments, other shapes for the tube body 10 or tube body 110 are contemplated. For example, a prosthetic tear duct device 1 or 100 can have a tube body 10 or 110 that has an alternate outer body shape, including ovular, elliptical, polygon prism (e.g., rectangular prism), conical, an irregular (e.g., an abstract shape), curved shape, a wavy shape (e.g., sine wave shape), an angular shape, etc. A non-limiting example of an angular shape is an elbow shape. The prosthetic tear duct device 1 or 100 described herein can have the first end portion 16 or 116 and/or the second end portion 18 or 118 of the tube body 10 or 110 with an opening having various shapes. Non-limiting examples of the opening of the first end portion 16 or 116 or the second end portion 18 or 118 include a bevel opening, a narrow opening (e.g., a funnel), and expanded opening (e.g., the first end potion 16 or 116 has and/or the second end portion 18 or 118 as a greater circumference than the tube body), or have an alternate shape (e.g., polygon, elliptical, oval, or circular).

The first end portion 16 or 116 and/or second end portion 18 or 118 of the tube body 10 or 110 could be composed of rigid or flexible materials, including but not limited to various forms of glass, metals, plastics, and other composites. The prosthetic tear duct device 1 or 100 described herein could include an attachable and detachable plug to cover/close the opening of the first end portion 16 or 116 and/or the second end portion 18 or 118 of the tube body 10 or 110. The prosthetic tear duct device 1 or 100 described herein can have a coating. In some embodiments, the prosthetic tear duct device 1 or 100 can include a frosted coating. In some embodiments, the prosthetic tear duct device 1 or 100 can include a microporous high-density polyethylene coating (e.g., MEDPOR®). The prosthetic tear duct device 1 or 100 described herein can include one or more suture holes.

The one-way valve 25 can be positioned outside of the tube body 10 proximate to the first end portion 16 and/or the second end portion 18. For example, the one-way valve 25 can be positioned outside of the tube body 10 and configured to contact the first end portion 16 and/or the second end portion 18.

In some embodiments, an anchor member 14 is located on the interior of the tube body 10 or 110 and deployable to increase the circumference of the tube body 10 or 110 such that the exterior of the tube body 10 or 110 contacts an anatomical region (e.g., nasolacrimal tissue). For example, an inflatable device (e.g., a balloon) positioned on an internal portion of the tube body 10 or 110 can be selectively filled with fluid (e.g., air, liquid, etc.) to increase the circumference of the tube body 10 or 110. The increased circumference of the tube body 10 or 110 can secure the prosthetic tear duct device 1 or 100 in place by friction between the exterior portion of the tube body 10 or 110 and the anatomical region. In some embodiments, the balloon could be deflated to allow for removal.

The prosthetic tear duct device 1 or 100 described herein can include a rotational mechanism in which rotation with a tool can induce expansion of the tube 10 or 110 circumference to induce a tight fit between the tube body 10 or 110 and the anatomical region.

In some embodiments, the prosthetic tear duct device 1 or 100 can comprise one or more pieces that interconnect. In some examples, the one or more pieces can be inserted from the medial canthus end of the tear duct channel, from the nose end of the tear duct channel, or from both ends of the tear duct channel, either concurrently or in separate maneuvers.

The prosthetic tear duct device 1 or 100 described herein can have additional flanges located along the tube body 10 or 100 or located on either end of the tube body 10 or 100 (e.g., first end portion 16 and second end portion 18). The prosthetic tear duct device 1 or 100 described herein can include one or more, or zero flanges. The prosthetic tear duct device 1 or 100 described herein can have one or more flanges of different sizes, materials, and/or shapes. Flanges (e.g., external flange 12, flange 30, flange 32, or external flange 112) can be composed of rigid or flexible materials, including but not limited to various forms of glass, metal, plastics, and other composites. A flange 12, 30, 32, or 112 can vary in size, material, and/or shape relative to other flanges 12, 30, 32, or 112 attached to the same prosthetic tear duct device 1 or 100. An angle in which flanges 12, 30, 32, or 112 are positioned can vary, both between prosthetic tear duct device 1 or 100 designs and between flanges 12, 30, 32, or 112 attached to a single device. One or more flanges 12, 30, 32, or 112 can be stationed anywhere on the tube body 10 or 110. A flange 12, 30, 32, or 112 can be shaped as several projectile members of any number radiating around the tube body 10 or 100. For example, one or more flanges 12, 30, 32, or 112 can be stationed in a spiral pattern around the tube body 10 or 110. The prosthetic tear device 1 or 100 described herein can have a single flange, multiple flanges (e.g., external flange 12, flange 30, and/or flange 32), or zero flanges. In some embodiments, the prosthetic tear device 1 or 100 does not include external flange 12 or 112.

In some embodiments, the prosthetic tear device 100 can include additional flange (e.g., flange 30 and/or flange 32). In some embodiments, the flanges of the prosthetic tear device 100 can be a balloon that can be inflated and deflated as needed for insertion or removal of the prosthetic tear duct device 100. For example, an additional flange (e.g., flange 30) can be comprised of a balloon that can be in a deflated configuration when the prosthetic tear device 100 is inserted into an anatomical region and filled with fluid when in an inflated configuration to hold the prosthetic tear device 100 in place. In another example, an additional flange (e.g., flange 32) can be comprised of a balloon that can be in a deflated configuration when the prosthetic tear device 100 is inserted into an anatomical region and filled with fluid when in an inflated configuration to hold the prosthetic tear device 100 in place. In another example, the external flange 112 and any additional flanges (e.g., flange 30 and/or flange 32) can each be comprised of a balloon that can be in a deflated configuration when the prosthetic tear device 100 is inserted into an anatomical region and filled with fluid when in an inflated configuration to hold the prosthetic tear device 100 in place.

The prosthetic tear duct device 1 or 100 described herein can utilize multiple types of anchor members 14. For example, a prosthetic tear duct device 1 or 100 can utilize shaped protrusions 150 in combination with anchor members 14.

The prosthetic tear duct device 1 or 100 described herein can include an alternate anchor member 14. For example, a hole drilled in an anatomical region to create a cavity (e.g., the alternate tear duct canal) can include ridges for a screw-like device to screw into the anatomical region with the prosthetic tear duct device 1 or 100 described equipped with said screw ridges on the outside of the tube body 10 or 110. The tube body 10 or 110 could then be screwed into the screw-ridged hole.

In an alternate embodiment, anchor members 14 can be barbs having a curved shape. For example, anchor members 14 can be composed of a semi-flexible material, allowing them to embody a linear position when retracted and a curved shape when deployed. The prosthetic tear duct device 1 or 100 described herein can include an interior, exterior, or in-hole lining of anchor member 14 holes to ensure a seal between the anatomical region and the longitudinal lumen 22, 122, or 123. In other examples, the anchor members 14 are stationary. For example, the anchor members 14 can have deployment capability but lack retraction capability. In other embodiments, the anchor members 14 could have retraction capability but lack deployment capability.

The prosthetic tear duct device 1 or 100 described herein could include alternate anchor member 14 retraction and deployment mechanisms, including but not limited to a sliding mechanism that includes a ramp surface, rotational mechanism, or balloon inflation mechanism. In one aspect, a rotational mechanism can be used to deploy the prosthetic tear duct device 1 or 100 via a deployment tool 31 used to engage with a rotational inner cylinder with a complementary shape relative to the deployment tool 31 to facilitate rotation by manually applying torque to the deployment tool 31. For example, the rotation of an inner cylinder in a first direction can cause an inner mechanism to deploy one or more anchor mechanisms 14. The rotation of the inner cylinder in an opposite direction can cause the inner mechanism to retract the one or more anchor mechanisms 14. In some embodiments, a locking mechanism can maintain the rotational cylinder position, which can selectively reconfigure that the anchor members 14 are in either the deployed or the retracted positions.

Alternate tools can be used to position the prosthetic tear duct device 1 or 100, deploy anchor members 14, or retract anchor members 14. For example, the prosthetic tear duct devices 1 and 100 described herein can use a first tool (e.g., deployment tool 31 of FIG. 9 and/or retraction tool 39 of FIG. 10A and 10B) to deploy or retract anchor members 14 of prosthetic tear duct device 1 or 100 and a second tool to position the prosthetic tear duct device 1 or 100. In some embodiments, the prosthetic tear duct device 1 or 100 described herein can be positioned by the same tool used to deploy and/or retract anchor members 14. Tools as described herein can vary in shape and function depending on which anchor member 14 is used by the prosthetic tear duct device 1 or 100.

It is to be understood that while the invention has been described in conjunction with the detailed description thereof, the foregoing description is intended to illustrate and not limit the scope of the invention. Other aspects, advantages, and modification are within the scope of the following claims.

Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

What is claimed is:

1. A prosthetic tear duct system, comprising:
a body comprising first end portion and a second end portion, the body defining a longitudinal lumen extending between the first and second end portions;
one or more anchor members movably coupled to the body, each anchor member being selectively reconfigurable from a retracted position to a deployed position,
wherein tip end portions of the one or more anchor members extend radially outward from an outer surface of the body when the one or more anchor members are in the deployed positions, and
wherein, when the one or more anchor members are in the retracted positions, the one or more anchor members are each radially retracted in comparison to the deployed positions; and
a one-way valve within the longitudinal lumen.

2. The system of claim 1, wherein the one-way valve is one of a shut-off valve, a ball-and-spring-valve, a compression stop valve, a compression stop valve, swing check valve, tilting disc check valve, reed valve, diaphragm check valve, or a duckbill valve.

3. The system of claim 1, wherein the one-way valve is configured to allow passage of fluid through the longitudinal lumen in a first direction from the first end portion to the second end portion and to prevent fluid from passing through the longitudinal lumen in a second direction from the second end portion to the first end portion.

4. The system of claim 1, wherein the one or more anchor members comprise a plurality of anchor barbs that are individually selectively reconfigurable from the retracted position to the deployed position.

5. The system of claim 4, wherein the plurality of anchor barbs are individually selectively reconfigurable from the deployed position to the retracted position.

6. The system of claim 5, further comprising a tool configured for manual reconfiguration of the plurality of anchor barbs between the deployed positions and the retracted positions.

7. The system of claim 1, further comprising a valve-housing unit comprising the one-way valve within the longitudinal lumen.

8. The system of claim 1, wherein the body is a cylindrical tube, an oval tube, a conical tube, a curved tube, or a polygon prism.

9. The system of claim 1, wherein the longitudinal lumen has an hourglass shape.

10. The system of claim 1, wherein the first end portion of the body of the prosthetic tear duct apparatus comprises a flange.

11. A prosthetic tear duct apparatus comprising:
a body comprising a first end portion and a second end portion, the first end portion comprising a first flange, the body defining a longitudinal lumen extending between the first and second end portions; and
a plurality of rings extending from an outer surface of the body and configured to contact nasolacrimal tissue,
wherein the longitudinal lumen has an hourglass shape.

12. The apparatus of claim 11, further comprising a one-way valve within the longitudinal lumen.

13. The apparatus of claim 12, wherein the one-way valve is one of one of a shut-off valve, a ball-and-spring-valve, a compression stop valve, a compression stop valve, swing check valve, tilting disc check valve, reed valve, diaphragm check valve, or a duckbill valve.

14. The apparatus of claim 13, further comprising a valve-housing unit proximal to the second end portion of the body configured to contain the one-way valve, wherein the one-way valve is configured to allow passage of fluid through the longitudinal lumen in a first direction from the first end portion to the second end portion and prevent fluid from passing through the longitudinal lumen in a second direction from the second end portion to the first end portion.

15. The apparatus of claim 11, wherein the second end portion comprises a second flange.

16. The apparatus of claim 15, wherein the second flange has a smaller circumference than the first flange.

17. The apparatus of claim 11, further comprising a selectively removable plug that is configured to couple to the first end portion, the second end portion, or both.

18. A method of manufacturing a tear duct apparatus, comprising forming a tube body comprising:
a first end portion and a second end portion, the first end portion comprising a first flange, the tube body defining a longitudinal lumen extending between the first and second end portions; and
a plurality of rings extending from an outer surface of the tube body and configured to contact nasolacrimal tissue,
wherein the longitudinal lumen has an hourglass shape.

19. The method of claim 18, wherein the tube body is made of glass, metal, plastic, rubber, latex, or combinations thereof.

20. The method of claim 18, wherein the tube body includes a frosted coating, a microporous high-density polyethylene implant coating, or both.
